(19) United States
(12) Patent Application Publication
(10) Pub. No.: US 2004/0043377 A1
(43) Pub. Date: Mar. 4, 2004

(54) Evaluation method for predicting pharmacokinetics of PM using PM liver cells of drug metabolozing enzyme cytochrome P450 having a genetic polymorphism

(75) Inventors: Kaoru Shimada, Aichi-ken (JP); Tadayuki Takashima, Aichi-ken (JP)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 10/652,752

(22) Filed: Aug. 28, 2003

(30) Foreign Application Priority Data
Aug. 30, 2002 (JP) 2002-255626

(51) Int. Cl. C12Q 1/00; C12Q 1/68

(52) U.S. Cl. 435/4; 435/6

(57) ABSTRACT

There is provided a novel evaluation method for predicting the pharmacokinetics of PM using PM liver cells of drug metabolizing enzyme cytochrome P450 having a genetic polymorphism. According to the present invention, the pharmacokinetics (metabolism) of PM can be predicted by using PM liver cells of CYP2D6among drug metabolizing enzyme cytochrome P450 known to have a genetic polymorphism.

### FOREIGN PRIORITY

The present application claims priority from Japanese Patent Application Number JP 2002-255626, filed Aug. 30, 2002.

### FIELD OF THE INVENTION

The present invention relates to an evaluation method for predicting pharmacokinetics of PM using PM liver cells of a molecular species of cytochrome P450 having a genetic polymorphism. More particularly, the present invention relates to an evaluation method for predicting pharmacokinetics of PM using PM liver cells having the above-mentioned genetic polymorphic CYP2D6, CYP2C9 or CYP2C19, and a kit therefore.

### BACKGROUND OF THE INVENTION

Although drug interaction presents a significant problem in the clinical setting, a typical cause of this drug interaction involves inhibition and induction of drug metabolizing enzymes by concomitant drugs. The early detection of these problems and the avoidance or risk are extremely important from the viewpoint of the proper use of pharmaceuticals.

Pharmacokinetics and toxicological characteristics, together with pharmacological activity, are decisive factors for the success of a candidate drug in the clinical setting. Since tremendous amounts of costs and time are required relating to clinical studies, it would be ideal if pharmacological and toxicological characteristics in humans could be determined prior to clinical studies for drug development. Although pre-clinical studies can be conducted using non-human laboratory animals, there are numerous cases in which results obtained using laboratory animals cannot be used to predict the results of clinical studies in humans with respect to the pharmacological and toxicological effects of xenobiotics due to interspecies differences in biotransformation.

The major cause of interspecies differences in the biotransformation of xenobiotics is drug metabolizing enzymes, and more particularly, differences in isoforms containing a genetic polymorphism of cytochrome P450 (CYP). Since the liver is the primary organ for drug metabolism, experimental systems originating in human liver have been used for evaluating human-specific drug characteristics. These human liver experimental systems consist of those that use cells such as liver cells and liver slices, as well as acellular systems, examples of which include homogenates, S9, microsomes and cytosols.

A. Guillouzo et al., Chemico-Biological Interactions, 121 (1999), 7-16 describes that cryopreserved storage in liquid nitrogen is preferable for storage of isolated liver cells for extended periods of time. This document does not contain any description regarding the evaluation of pharmacokinetics of PM using liver cells.

A. P. Li et al., Chemico-Biological Interactions, 121 (1999), 17-35 describes a comparison between cryopreserved liver cells and non-cryopreserved liver cells using kinetic analysis. This document does not contain any description regarding the evaluation of pharmacokinetics of PM using liver cells.

A. P. Li et al., Chemico-Biological Interactions, 121 (1999), 117-123 describes the usefulness of liver cells. This document relates to a test using liver cells, and although it lists evaluation of metabolism, toxicity, drug interactions, enzyme induction and the effects of cytokines and hormones and so forth, there is no description whatsoever regarding being able to evaluate pharmacokinetics of PM using liver cells.

Chladeck, J. et al., Eur. J. Clin. Pharmacol. (2000) 56: 651-657 describes that dextromethorphan is widely used as a probe for evaluating the activity of cytochrome P450 2D6 (CYP2D6) in vivo, and the results of comparing the metabolic ratios from DM to dextrorphan (DEX) in urine and plasma in healthy Caucasians.

As has been indicated above, a system for evaluating the pharmacokinetics of PM using PM liver cells was not known prior to filing of the present application.

A simple in vitro evaluation system is desired that enables preliminary evaluation of the human pharmacokinetics of PM.

Therefore, the inventor of the present invention began metabolic tests using isolated human liver cells to solve the above problems.

Drug metabolizing enzyme cytochrome P450 is known to have genetic polymorphism, and in the clinical setting, is one of the causes of the occurrence of considerable variations in the appearance of toxicity and pharmacological efficacy attributable to differences in metabolic function. Namely, among cytochromes P450 known to have genetic polymorphisms, studies were conducted on whether or not the pharmacokinetics (metabolism) of PM can be predicted using PM cryopreserved isolated human liver cells in which activity is remarkably low as a result of a missing or mutated CYP2D6 gene.

### SUMMARY OF THE INVENTION

As a result of repeated studies as described above, the inventor of the present invention found that, among drug metabolizing enzyme cytochromes P450 known to have genetic polymorphisms, the use of cryopreserved PM isolated liver cells of CYP2D6 make it possible to predict the pharmacokinetics (metabolism) of PM, thereby leading to completion of the present invention.

Namely, in a first aspect of the present invention, an evaluation method is provided for predicting pharmacokinetics of PM comprising: reacting PM liver cells of a molecular species of cytochrome P450 having a genetic polymorphism, with a test compound in a culture liquid.

In the above evaluation method, the reaction is allowed to proceed by culturing the culture liquid at a prescribed temperature and for a prescribed period of time followed by kinetic analysis.

The genetic polymorphism of cytochrome P450 can be selected from the group consisting of CYP3A4, CYP3A5, CYP3A7, CYP2D6, CYP2C9, CYP2Cl9, CYP2A6, CYP1A1, CYP1A2 and CYP2E1. The genetic polymorphism of cytochrome P450 is preferably selected from the group consisting of CYP2D6, CYP2C9 and CYP2C19. The genetic polymorphism of cytochrome P450 may also be CYP2D6.

There are no particular restrictions on the above liver cells of human PM, and suspended liver cells or adhered liver cells on culture plate may be used.

Although there are no particular restrictions on the reaction temperature, it is preferably near body temperature at 36.5° C. to 37.5° C.

Although there are no particular restrictions on the reaction time, and may differ depending on the test compound. It is preferably within 4 hours, and more preferably within 2 hours. The reaction may be stopped by sampling at desired times (for example, 0 minutes, 0.5 hours, 1 hour and 2 hours).

The culture liquid may be a typically used culture liquid such as Krebs Henseleit buffer, and a suitable carrier may be added. Namely, any culture liquid may be used as long as it does not have an effect on enzyme activity.

In another aspect of the present invention, a kit is provided for use in the above evaluation method for predicting pharmacokinetics of PM comprising: PM liver cells having a genetic polymorphism of cytochrome P450, and a culture liquid.

When used in the present specification, the term “PM (Pool Metabolizer) of a molecular species of cytochrome P450′ refers to humans in which enzyme activity of a certain specific molecular species of cytochrome P450 is lower than in normal humans due to gene mutations or deletion or regulation of expression and so forth. At present, although known examples of genetic polymorphisms include CYP3A4, CYP3A5, CYP3A7, CYP2D6, CYP2C9, CYP2C19, CYP2A6, CYP1A1, CYP1A2 and CYP2E1, since there is the possibility of the discovery of cytochromes having new genetic polymorphisms in the future, the pharmacokinetics of PM of each genetic polymorphisms can be predicted with the method of the present invention in such cases as well.

### BRIEF DESCRIPTION OF THE DRAWINGS

FIG. 1 is a schematic drawing showing the metabolic pathway of dextromethorphan (DM).

FIG. 2 is a graph representing the relationship between dextromethorphan (DM) concentration and dextrorphan (DEX) formation rate (pmol/min/10⁶ cells) in PM isolated human liver cells and EM isolated human liver cells.

FIG. 3 is a graph representing the relationship between dextromethorphan (DM) concentration and 3-methoxymorphinan (3-MM) formation rate (pmol/min/10⁶ cells) in isolated PM liver cells and isolated EM liver cells.

FIG. 4 is a graph representing the relationship between dextromethorphan (DM) concentration and dextrorphan conjugate (DEX-glucuronide) formation rate (pmol/min/10⁶ cells) in isolated PM liver cells and isolated EM liver cells.

FIG. 5 is a graph representing the relationship between dextromethorphan (DM) concentration and 3-MM formation rate divided by 1′OH-MDZ formation rate in PM liver cells (hepatocytes) and EM liver cells.

FIG. 6 is a graph representing the relationship between dextromethorphan (DM) concentration and 3-MM formation rate divided by 1′OH-MDZ formation rate in PM liver microsomes and EM liver microsomes.

### DETAILED DESCRIPTION OF THE INVENTION

When used in the present specification, the term “kinetic analysis” refers to an analysis of the elimination rate of a test compound, the formation rate of a metabolite and so forth by mathematical and statistical techniques.

As is indicated in the following examples, in the present invention, whether or not the pharmacokinetics (metabolism) of PM can be predicted using PM cryopreserved human isolated liver cells of CYP2D6 among drug metabolizing cytochromes P450 for which genetic polymorphisms are known, was examined by using dextromethorphan (DM), a substrate of CYP2D6, as a probe.

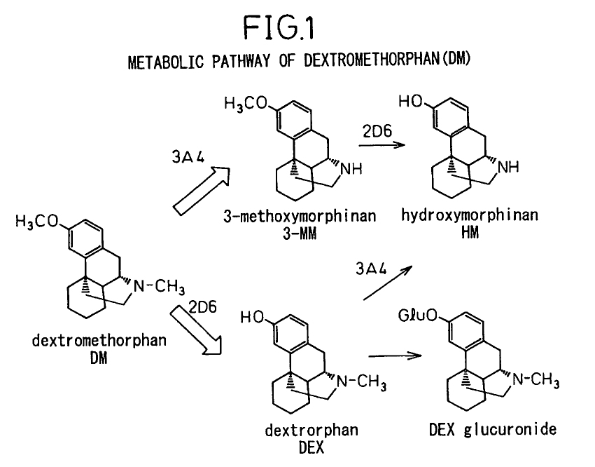

The major metabolic pathway of dextromethorphan (DM) is shown in FIG. 1. In the clinical setting, metabolism of dextromethorphan (DM) is known to proceed by N-demethylation, O-demethylation and subsequent glucuronic acid conjugation. CYP3A4 catalyzes the N-demethylation reaction, while CYP2D6 catalyzes the O-demethylation reaction. In the case of EM (Extensive Metabolizers) of CYP2D6 having normal metabolic function, O-demethylation metabolism mainly proceeds by CPY2D6, while there is no major metabolic pathway for N-demethylation by CYP3A4. On the other hand, in the case of PM of CYP2D6, since the metabolic function of CYP2D6 is insufficient, N-demethylation is known to primarily proceed as a compensatory metabolic pathway of O-demethylation. In the evaluation system as claimed in the present invention as well, a compensatory metabolic reactions were observed that are similar to clinical results. Thus, if PM liver cells of a molecular species of cytochrome P450 are used, and the above molecular species and a test compound are allowed to react in a culture liquid, the pharmacokinetics of PM of each molecular species of cytochrome P450 can be predicted.

As has been described above, an evaluation system that uses PM liver cells is not known in the prior art. Thus, since the metabolic evaluation test using PM liver cells as claimed in the present invention is able to ascertain the human pharmacokinetics (metabolic pattern) of PM prior to clinical studies, it can be said to be a widely applicable evaluation method that is extremely useful for enhancing the predictability of clinical studies.

Although drug metabolizing enzyme cytochrome P450 is known to have several other genetic polymorphisms besides CYP2D6, examples of which include CYP3A4, CYP3A5, CYP3A7, CYP2C9, CYP2C19, CYP2A6, CYP1A1, CYP1A2 and CYP2E1, PM liver cells of these genetic polymorphisms can also be used in the evaluation system as claimed in the present invention in the same manner as those of CYP2D6.

Although PM liver cells of CYP2D6 are used in the examples described below, the following lists examples of other molecular species besides CYP2D6 that can be used in evaluation systems of the pharmacokinetics of PM.

The evaluation method as claimed in the present invention can be carried out by a method similar to the following examples by selecting for the test compound of CYP2C19, for example, S-mephenytoin or omeprazole, and using cryopreserved PM liver cells of CYP2C19. Known methods can be used for measuring the unchanged form and metabolites. Lot HH-092, HH-016 or HH-023 and so forth prepared at In Vitro Technologies (IVT), USA can be used for the cryopreserved PM liver cells of CYP2C19.

The evaluation method as claimed in the present invention can be carried out by a method similar to the following examples by selecting for the test compound of CYP1A2, for example, ethoxyresorufin or caffeine, and using cryopreserved PM liver cells of CYP1A2. Known methods can be used for measuring the unchanged form and metabolites.

The evaluation method as claimed in the present invention can be carried out by a method similar to the following examples by selecting for the test compound of CYP2C09, for example, phenytoin, tolubutamide, ibuprofen, diclofenac, warfarin or naproxen, and using cryopreserved PM liver cells of CYP2C9. Known methods can be used for measuring the unchanged form and metabolites. Lot HH-046, HH-056, HH-099, HH-114, HH-GUY, HH-WWM and so forth prepared at IVT can be used for the cryopreserved PM liver cells of CYP2C9.

The evaluation method as claimed in the present invention can be carried out by a method similar to the following examples by selecting for the test compound of CYP2A6, for example, coumarin or nicotine, and using cryopreserved PM liver cells of CYP2A6. Known methods can be used for measuring the unchanged form and metabolites.

The evaluation method as claimed in the present invention can be carried out by a method similar to the following examples by selecting for the test compound of CYP2E1, for example, chlorzoxazone or acetominophen, and using cryopreserved PM liver cells of CYP2E1. Known methods can be used for measuring the unchanged form and metabolites.

The evaluation method as claimed in the present invention can be carried out by a method similar to the following examples by selecting for the test compound of CYP3A, for example, midazolam, nifedipine or testosterone, and using cryopreserved PM liver cells of CYP3A. Known methods can be used for measuring the unchanged form and metabolites.

Although the following provides a detailed explanation of the present invention with reference to the following examples and attached drawings, the scope of the present invention should not be interpreted as being limited thereby.

### EXAMPLES

### Example 1

### Method

A preliminary test for evaluation metabolic reactions was conducted by selecting dextromethorphan (DM, analgesic agent) as the substrate of CYP2D6. The major metabolic pathway of DM is shown in FIG. 1. Metabolism of DM in the clinical setting is known to proceed by N-demethylation, O-demethylation followed by glucuronic acid conjugation. CYP3A4 catalyzes the N-demethylation reaction, while CYP2D6 catalyzes the O-demethylation reaction. In PM of CYP3A4, N-demethylation is known to proceed as a compensatory metabolic pathway of O-demethylation. In the present embodiment, metabolic reactions were examined for dextromethorphan using isolated PM liver cells of CYP2D6, and a discussion was made as to whether or not the pharmacokinetics of PM can be predicted.

The cryopreserved isolated PM liver cells of PM of CYP2D6 that were used (Lot 64) and the cryopreserved isolated EM liver cells (Lot 70) were prepared at In Vitro Technologies (IVT), USA. Liver cells for which the activity of CYP3A4 was roughly equal to that of Lot 64 of the cryopreserved isolated PM liver cells were selected by referring to data provided by IVT for use as the cryopreserved isolated EM liver cells. The acquired cells consisted of 6×10⁶ cells per vial for both lots.

Krebs Henseleit buffer (adjusted to pH 7.4 following addition of calcium chloride dihydrate (0.373 g/L), sodium bicarbonate (2.1 g/L) and HEPES (1.5 g/L)) was used for the incubation medium. The cryopreserved human isolated liver cells were inoculated into a 96-well plate and used for testing while suspended in culture liquid. Dextromethorphan (DM), which is the substrate of CYP2D6, was added to the wells at final concentrations of 0.08, 0.4, 2, 10 and 50 μM and allowed to react at 37° C. The reaction liquid was sampled after 1 and 2 hours, and the parent compound (DM) along with dextrorphan (DEX: metabolite mainly produced by CYP2D6) and 3-methoxymorphinan (3-MM: metabolite mainly produced by CYP3A4) were respectively assayed. In addition, with respect to assay of the conjugate (glucuronide), DEX obtained by hydrolysis following the addition of β-glucuronidase/allylsulfatase to the sampled reaction liquid was assayed, and the difference with DEX prior to hydrolysis was taken to be the amount of the conjugate. LC/MS/MS were used for analyzing the unchanged form and metabolites in the culture liquid.

The respective CYP3A4 activities of isolated EM liver cells and isolated PM liver cells were compared (or normalized) by using as an indicator the 1′hydroxylation activity of midazolam (MDZ), which is thought to belong to the same substrate type of CYP3A4 as DM. The method of the metabolism test for CYP3A4 consisted of incubating MDZ in compliance with the method described above, and analyzing 1′-hydroxymidazolam (1′OHMDZ). LC/MS/MS were used in the same manner as described above for analyzing 1′-OH MDZ.

Furthermore, LC/MS/MS were carried out under the measurement conditions described below. HPLC: Waters 2790, MS: API365 Sciex, column: YMC J′ sphere ODS L80 2×35 mm, gradient: mobile phase A [CH₃CN:10 mM CH₃CO₂NH₄ aqueous solution=10:90], mobile phase B [CH₃CN:10 mM CH₃CO₂NH₄ aqueous solution=80:20], conditions [mobile phase flow rate 0.35 ml/minute, composition of mobile phase changed from A:B=100:0 to A:B=0:100 1 minute after sample injection, followed by allowing composition of A:B=0:100 to flow for 1 minute], MS/MS detection: DM=272.3/170.9, DEX=258.2/157.1, 3-MM=258.2/215.0, MDZ=326.1/291.1, 4-OH MDZ=342.1/324.1.

### Example 2

### Relationship Between Dextromethorphan (DM) Concentration and Dextrorphan (DEX) Formation Rate (pmol/min/10⁶ cells) in Isolated PM Liver Cells Lot 64 and Isolated EM Liver Cells Lot 70

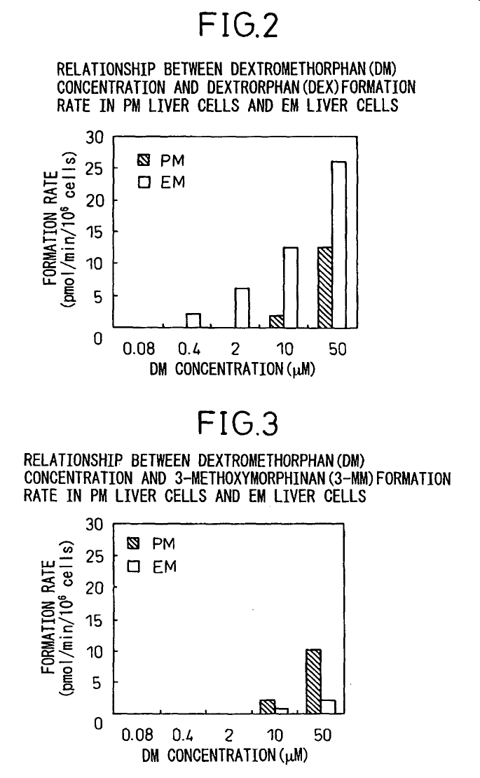

The relationship between dextromethorphan (DM) concentration and dextrorphan (DEX) formation rate (pmol/min/10⁶ cells) is shown in FIG. 2. The formation rates shown in FIG. 2 were determined from values calculated according to the amount formed after 1 hour.

DEX, which is a metabolite in PM liver cells, was not detected up to the concentration of Km (about 2 μM) for CYP2D6 of DM. As shown in FIG. 1, although DEX is formed from DM as a result of O-demethylation, this pathway is catalyzed by CYP2D6. Thus, in PM liver cells in which CYP2D6 is missing, the reaction of this metabolic pathway can be understood to have not occurred. Actually, there was only slight formation of DEX in PM even in the vicinity of concentration of DM in the blood (up to 1 μM) at the clinical dose level (about 20 mg/body). When the concentration was increased to 10 μM and 50 μM, the formation of DEX was observed in PM liver cells as well. In addition, in EM liver cells, the formation rate of metabolite DEX increased concentration-dependently.

### Example 3

### Relationship Between Dextromethorphan (DM) Concentration and 3-Methoxymorphinan (3-MM) Formation Rate (pmol/min/10⁶ cells) in Isolated PM Liver Cells Lot 64 and Isolated EM Liver Cells Lot 70

The relationship between the formation rate of 3-methoxymorphinan (3-MM), which is formed by N-demethylation catalyzed by CYP3A4, and dextromethorphan (DM) concentration is shown in FIG. 3. In EM liver cells, the formation rate of 3-MM was less than one-tenth that of the DEX formation rates shown in FIG. 2, and these results coincided with clinical results in humans. On the other hand, in PM liver cells, the formation rate of 3-MM was comparable to the DEX formation rates shown in FIG. 2. This supports the finding that, in PM of CYP2D6, N-demethylation proceeds in the form of a compensatory metabolic pathway as a result of the decrease of O-demethylation.

### Example 4

### Relationship Between Dextromethorphan (DM) Concentration and Dextrorphan Conjugate (DEX-glucuronide) Formation Rate (pmol/min/10⁶ cells) in Isolated PM Liver Cells Lot 64 and Isolated EM Liver Cells Lot 70

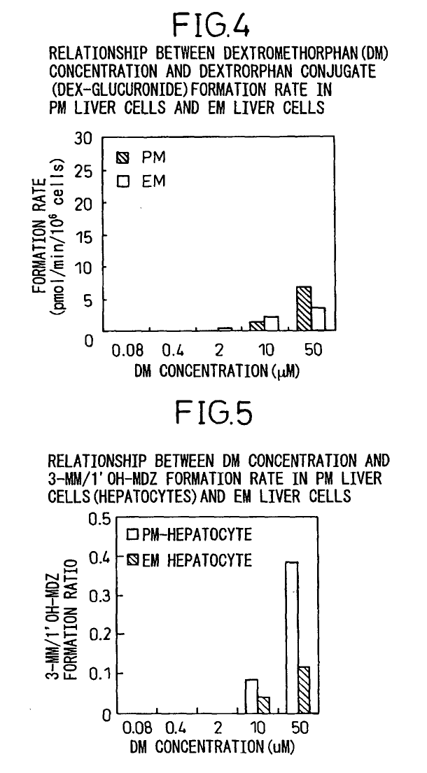

The results of analyzing dextrorphan conjugate are shown in FIG. 4. The glucuronic acid conjugation reaction was clearly determined to proceed for both isolated PM liver cells lot 64 and isolated EM liver cells lot 70.

### Example 5

### Comparison of CYP3A4 Activity in Isolated PM Liver Cells Lot 64 and Isolated EM Liver Cells Lot 70

The respective activities of isolated EM liver cells and isolated PM liver cells were compared (or normalized) using as an indicator the 1′-hydroxylation activity of midazolam (MDZ), which is thought to belong to the same substrate type as DM, in the manner previously described. Following incubation of MDZ in compliance with the method used for DM, the formation rates of 1′-hydroxymidazolam (1′OH-MDZ) were 26.6 and 18.2 (pmol/min/10⁶ cells) in isolated EM liver cells and isolated PM liver cells, respectively. There was roughly a 30% difference in the activity values (formation rates) between the liver cells, and even the metabolic results of EM and PM were respectively corrected using the above values, they were confirmed not to have an effect on the above discussion relating to metabolic behavior described in Examples 2 through 4.

### Example 6

### Method Using PM Liver Microsomes and EM Liver Microsomes

Incubation reactions from the determination of the enzymatic kinetic parameters were carried out at a protein concentration of 0.2 mg/ml in 100 mM potassium phosphate, pH 7.4 containing 1.3 mM NADP⁺, 0.93 mM NADH, 3.3 mM glucose-6-phosphate, 8 units/ml G-6-PDH, 3.3 mM MgCl₂, and substrates. Reactions were initiated by the addition of NADP⁺/NADH, and then terminated after 10 min incubation at 37° C. by the addition of ten-fold volume of acetonitrile containing an LC/MS/MS internal standard (Levallorphan at 100 ng/ml final conc.). The mixtures were centrifuged for 10 min, and supernatants were used for LC/MS/MS analyses to determine the production rates of metabolites.

### Example 7

### Comparison of CYP3A4 Activity and Formation Rates of DM Metabolites Using PM Liver Microsomes (Lot. No. HHM-0168) and EM Liver Microsomes

Product formation rates of DEX metabolites and midazolam metabolite in HLM were determined. 3-MM formations in EM microsomes (4.26 to 20.0 pmol/min/mg protein) were higher than those in PM microsomes (2.24 to 11.3 pmol/min/mg protein). Moreover, even in normalizing 3-MM formation by 1′OH-MDZ activity, 3-MM/1′OH-MDZ formation ratio in PM microsomes (0.0009-0.0477) was lower than that in EM microsomes (0.0126-0.0595). Little difference of 3-MM/1′OH-MDZ formation ratio between PM and EM microsomes were different from those observed between PM and EM hepatocytes.

It was found that a compensatory metabolic pathway did not proceed in a test method using PM microsomes. On the contrary, in case where PM liver cells were used, a compensatory metabolic pathway was observed in a similar manner to that observed clinically. This indicates that the present invention is useful.

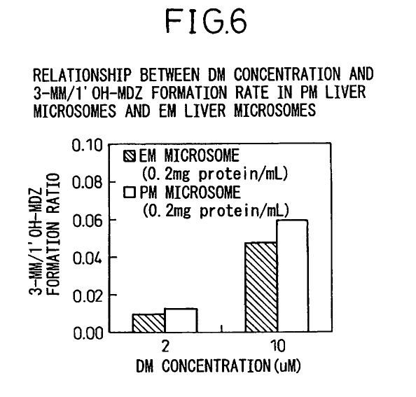

As shown in FIG. 5, after the product amounts of metabolite of DM, 3-MM, relative to 1′OH-MDZ was determined, differences in 3-MM/1′OH-MDZ formation rate were large between PM and EM liver cells (hepatocytes) in a similar manner to that observed clinically.

That is, in CYP2D6 PM liver cells, 3-MM formation rate was greater than that in EM liver cells, via a compensatory pathway of O-demethylation in a similar manner to that observed clinically. On the contrary, as can be seen form FIG. 6 wherein PM and EM liver microsomes were used instead of CYP2D6 PM and EM liver cells, differences in 3-MM/1′OH-MDZ formation rate were not statically significance. RESULT

As was described in Examples 2 and 3, as a result of measuring the formation rates of two types of metabolites of DM, namely 3-MM and DEX, a large difference in the ratio of the metabolites (3-MM/DEX) was observed between EM and PM. Namely, in PM liver cells of CYP2D6, compensatory metabolism of O-demethylation occurred in the same manner as clinical results, while the formation rate of 3-MM was larger than EM. In addition, the glucuronic acid conjugate of DEX was also observed. These findings qualitatively closely agree with clinical results in humans. Thus, the pharmacokinetics of PM was determined to be able to be predicted by using human PM isolated liver cells of CYP2D6. In the evaluation of the pharmacokinetics of test substances for which metabolism is unclear, the pharmacokinetics of PM can be predicted by kinetic analysis (for example, calculating the elimination rate of the test compound) under experimental conditions similar to the examples.

1. An evaluation method for predicting pharmacokinetics of PM comprising: reacting PM liver cells of a molecular species of cytochrome P450 having a genetic polymorphism, with a test compound in a culture liquid.

2. A method according to claim 1, wherein the reaction is allowed to proceed by culturing the culture liquid at a prescribed temperature and for a prescribed period of time followed by kinetic analysis.

3. A method according to claim 1, wherein the genetic polymorphism of cytochrome P450 is selected from the group consisting of CYP3A4, CYP3A5, CYP3A7, CYP2D6, CYP2C9, CYP2C19, CYP2A6, CYP1A1, CYP1A2 and CYP2E1.

4. A method according to claim 3, wherein the genetic polymorphism of cytochrome P450 is selected from the group consisting of CYP2D6, CYP2C9 and CYP2C19.

5. A method according to claim 3, wherein the genetic polymorphism of cytochrome P450 is CYP2D6.

6. A kit for use in the evaluation method for predicting pharmacokinetics of PM according to claim 1 comprising: PM liver cells of a molecular species of cytochrome P450 having a genetic polymorphism and a culture liquid.
